(19) United States
(12) Patent Application Publication
(10) Pub. No.: US 2023/0263159 A1
(43) Pub. Date: Aug. 24, 2023

(54) PERACETIC ACID ANTIMICROBIAL COMPOSITION SYSTEM AND METHOD

(71) Applicant: Donald Griffith, Houston, TX (US)

(72) Inventor: Donald Griffith, Houston, TX (US)

(21) Appl. No.: 18/107,788

(22) Filed: Feb. 9, 2023

Related U.S. Application Data
(63) Continuation-in-part of application No. 17/410,613, filed on Aug. 24, 2021.
(63) Continuation-in-part of application No. 17/070,365, filed on Oct. 14, 2020.
(60) Provisional application No. 63/441,916, filed on Jan. 30, 2023.
(60) Provisional application No. 63/232,288, filed on Aug. 12, 2021.

(51) Int. Cl. A01N 37/16; A01N 1/02; A61L 2/18; A01N 65/00

(52) U.S. Cl. CPC A01N 37/16; A01N 1/0215; A01N 65/00; A61L 2/186

(57) ABSTRACT

An antimicrobial composition includes peracetic acid and witch hazel extract. The peracetic acid can be formed by combining and reacting the acetic acid and hydrogen peroxide, for example at the point of use. Alternatively, the peracetic acid may be formed separately and mixed with the witch hazel extract prior to use. The resulting peracetic acid and hydrogen peroxide are, thus, combined with the witch hazel extract prior to use. The combination of peracetic acid, hydrogen peroxide and witch hazel extract may be applied as an antimicrobial. The antimicrobial composition may be applied topically to various organs (e.g., skin, nose, ears, mouth, throat, urethra, urinary bladder, gastrointestinal organs) or instilled through an indwelling urinary catheter or applied or inserted as a topical additive or coating to devices or other organ surfaces or other medical devices, or on the skin of the body or surfaces of inanimate objects.

### CROSS-REFERENCE TO RELATED APPLICATIONS

The present application is a conversion of and has benefit of priority of the following application, which is co-pending and has at least one same inventor of the present application: U.S. Provisional Pat. Application No. 63441916, titled “Peracetic Acid Antimicrobial Composition System and Method,” filed Jan. 30, 2023. The present application is also a continuation in part and has benefit of priority of U.S. Pat. Application No. 17/070,365, titled “Antimicrobial Combinations System and Method”, filed on Oct. 14, 2020. The present application is also a continuation in part and has benefit of priority of U.S. Pat. Application No. 17/410,613, titled “Antimicrobial Bladder Additives System and Method”, filed on Aug. 24, 2021. which was a conversion of and has benefit of priority of U.S. Provisional Pat. Application No. 63/232,288, titled “Antimicrobial Bladder Additives System and Method”, filed Aug. 12, 2021. Priority applications No. 17/070,365 and 17/410,613 are co-pending and have at least one same inventor of the present application and are herein incorporated by this reference.

### TECHNICAL FIELD

The invention generally relates to antimicrobial compositions and their uses, and more particularly relates to antimicrobial compositions of peracetic acid combinations efficacious for bladder exposure and other antimicrobial actions.

### BACKGROUND

Antimicrobial compositions are critical to inhibit infections in the body. Infections are a major concern in delivery of healthcare. Infections can present serious complications to patients, even life-threatening consequences in some instances. Chronically disabled and aged patients are particularly vulnerable to such infections. Various precautions and remedies exist, most notably cleansing and antibiotics. Effectiveness of these may be limited, however, in certain conditions. New antimicrobial compounds as surface or fluid additives would be advantageous.

It is noteworthy that the World Health Organization [WHO] website reports that no new antimicrobial agents have been commercialized in the past 40 years. New antimicrobial compounds, therefore, are much needed. Moreover, antimicrobials that don’t have the concerns with resistance of antibiotics in extended use, would be quite advantageous.

Many patients suffer from microbial and viral infections. Aging and disabled individuals commonly suffer from conditions (e.g., neurological, physical or cognitive) that are particularly susceptible to infections, for example, to urinary bacterial colonizations and or other complications that arise as consequence of one or more disabling conditions and the management of the disabled condition. Normal, periodic, volitional urinary bladder continence is often disrupted in the aging and disabled. Aging and childbirth can weaken the pelvic floor muscles in females causing incontinence issues. In males over the age of about 50, testosterone production slowly enlarges the prostate gland and slowly constricts and closes the urethral channel within the ‘donut-shaped’ prostate gland leading to urinary retention (i.e., inability to urinate). As can be understood, certain disabilities also may cause incontinence concerns. Many organs undergo similar aging-induced changes that make the organ susceptible to colonization by all types of microbial agents (e.g., bacteria, viruses and fungi).

Surgical procedures, such as transurethral resection of prostate gland, radical prostatectomy, pelvic floor reconstruction, use of artificial urinary sphincters, and others, may successfully rehabilitate bladder and urethral channel functions in some patients. Moreover, pads, pull-ups, and diapers (e.g., used mostly by females, but also by males), or indwelling urinary catheters (e.g., used mostly by males, whose use may involve suprapubic (abdominal) or urethral catheters), can be helpful for incontinence or urinary retention. Urinary catheter users develop catheter associated urinary tract infections (“CAUTI”) at a rate of approximately 5% of users/day. Thus, virtually all chronic urinary catheter users have chronic CAUTI. The National Institutes of Health (NIH), Center for Disease Control (CDC) and Food and Drug Administration (FDA) discourage preventive use of antibiotics in asymptomatic urinary catheter-using patients but favor use of such agents when urinary colonization is symptomatic. Antibiotics commonly lead to mutant ‘resistant’ strains of urinary pathogens. These and other options, nevertheless, can lead to symptomatic, life-altering and life-threatening infection problems.

In patients with long-term catheter use (e.g., typically 15+ days), catheter associated urinary tract infections are particularly a problem. NIH, CDC, and FDA have all identified CAUTI as one of the most common and expensive infectious diseases in the United States. Prophylactic use of antibiotics to treat these infections has been discouraged, because of evolution of antibiotic-resistant microbes. Microbial colonization risks of indwelling catheters, therefore, remain an unsolved clinical problem in a growing, aged population. The infection risks are similar regardless of whether the catheter is placed abdominally or transurethrally.

Causes of CAUTI include microbial attachment of skin microbes to external surfaces of indwelling catheters, followed by explosive growth and concurrent biofilm formation. Microbes commonly adhere to cellular and inanimate surfaces and then create and enshroud themselves within a protective biofilm. Daily migration of colony-rich biofilms, principally on catheter external surfaces toward the bladder lumen, induces colonization of bladder urine. Colonized bladder urine occurs at a rate of approximately 5% of catheter users per day. Thus, virtually 100% of chronic-use catheters have colonization after ~20+ days of indwelling catheter use. Consequently, long-term catheter users typically have colonized urine for as long as the catheter is indwelling.

It is noteworthy that urine is the excretory pathway for muscle (i.e., proteinaceous) and bone (i.e., calcium and phosphorus) waste resulting in high urinary concentrations of urea and high concentrations of calcium and phosphorus dissolved in urine. Some bacterial species (e.g., all Proteus species and some strains of Klebsiella and Pseudomonas) make urease, an enzyme that interacts with urea to robustly and quickly alkalinize urine. Alkaline urine triggers precipitation of calcium-phosphate crystals which over a few days attach and grow onto catheter surfaces as encrustations and/or as bladder stones.

Use of systemic antibiotics and antibiotic catheter coatings has been discouraged, for example, by FDA, NIH and CDC, because multiple trials have shown little continued sterility and frequent development of mutant microbial species that are or become resistant to antibiotics. It has become apparent that many of the microbial species that cause CAUTI owe much of their pathogenicity to quorum signaling and biofilm formation. Quorum signaling is a process in which bacteria communicate with each other by secreting and sensing diffusible signaling molecules called “autoinducers.” When these signaling molecules exceed a threshold concentration level, they auto-stimulate genes that enable bacteria to behave as a multicellular population, to benefit their survival. Staphylococci bacteria, as an example, create autoinducers that upregulate the expression of multiple toxins and enhance the formation of biofilms. These phenotypes are the hallmark of staph pathogenesis and are a cause of resistant and persistent infections and even death.

Consequently, a significant and serious need exists for suitable new antimicrobial compounds and uses thereof. It would, therefore, be a significant improvement in the art and technology to provide new microbiocidal compounds and uses thereof. It would further be an improvement to provide new microbiocidal compounds for bladder rinsing, as well as mucous and particulate cleansing to use with urinary catheters, as well as other devices. It would also be a further improvement to provide new or enhanced antimicrobial compounds and/or new or revised methods of use that do not significantly lead to resistant microbes and/or overuse of conventional antibiotics and similar conventional antimicrobial options.

### SUMMARY

An embodiment of the invention is a biological or inanimate surface cleansing, rinsing or coating antimicrobial composition. The composition includes peracetic acid and witch hazel extract.

Another embodiment of the invention is a reactive composition including acetic acid and hydrogen peroxide. The combination reacts to form peracetic acid and oxygen.

Yet another embodiment of the invention is a method of preventing microbial proliferation. The method includes providing acetic acid, providing hydrogen peroxide, reacting the acetic acid and the hydrogen peroxide to obtain peracetic acid, and providing witch hazel extract to the peracetic acid.

Another embodiment of the invention is a process for treating the surface of a medical device. The process includes exposing the medical device to a combination of peracetic acid and witch hazel extract.

Yet another embodiment of the invention is a process for treating an organ. The process includes exposing the organ to a combination of peracetic acid and witch hazel extract.

Another embodiment of the invention is a product medical device of the process for treating the surface of the medical device.

Yet another embodiment of the invention is a product organ of the process for treating the organ.

### DETAILED DESCRIPTION

Embodiments include peracetic acid compositions, as well as other formulations. As non-exclusive example, embodiments also can include organic extracts, such as Witch hazel extracts (WH), comprising hamamelitannin and/or gallic acid, combined with the peracetic acid, hydrogen peroxide and other constituents. The novel combination has been determined to be effective as an antimicrobial, for topical applications, including to surfaces of urinary catheters and devices, and as a internal rinse of the bladder lumenal mucosa, skin and other organs. Through benchtop experimentation, the peracetic acid+WH combination has been shown to be more effective as an antibacterial than the conventional Betadine solution. The formulations may take a variety of forms, such as the peracetic acid may be formed by reacting hydrogen peroxide with acetic acid at point of use, and may therefore include liquids, rinse, sprays, ointments, creams, colloids, and others.

### Peracetic Acid

Peracetic acid (CH₃CO₃H) has been found to be a potent antimicrobial agent. Peracetic acid may be made by combining hydrogen peroxide (H₂O₂) and acetic acid (CH₃CO₂H). Acetic acid (3%) and water (97%) are commercialized as vinegar. Hydrogen peroxide (2%) in water is also commercialized as a cleansing agent. By mixing the two compounds, hydrogen peroxide and acetic acid, peracetic acid and water is obtained, generally as follows:

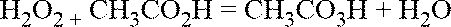

Peracetic acid is, thus, an organic compound with component bonds as follow:

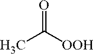

It is a colorless liquid with a characteristic acrid odor and can be corrosive.

The U.S. Environmental Protection Agency has registered peracetic acid as an antimicrobial for indoor use on hard surfaces. Peracetic acid is also registered for use in dairy and cheese processing, on food processing equipment, and in pasteurizers in breweries, wineries and beverage plants. It has also been used for disinfection of medical supplies, to prevent biofilm formation in pulp industries, and as a water purifier and disinfectant. The acid has further been used as a cooling tower water disinfectant to prevent biofilm formation.

Although peracetic acid has been used in medical context, formulations for biocidal decontamination and bactericides used both internally and externally in the human body have become possible when combined in select antibacterial formulations in accordance with teachings herein.

### Witch Hazel Extract

Combination of acidic aqueous combinations of peracetic acid with witch hazel concentrate has been found to be a very effective formulation for antimicrobial effects.

Witch hazel (Hamamelis) is a genus of flowering plants in the family Hamamelidaceae. Four species are found in North America (H. ovalis, H. virginiana and H. vernalis), and one species each is found in Japan (H. japonica) and China (H. mollis). The leaves and bark of the North American witch hazel, Hamamelis virginiana, may be used to produce an astringent decoction. This witch hazel decoction can be an extract in liquid form. This concentrated extract liquid has been FDA approved, for example, for topical application to the nose, anus and skin.

Witch hazel liquid extract/concentrate can include such components as calcium oxalate, gallotannins, and safrole, and chemicals found in the essential oil (carvacrol, eugenol). Witch hazel for use as a topical can be a liquid, semisolid ointment, cream, gel or salve, as examples. Witch hazel extracts and concentrates are widely sold by pharmacies, grocery stores, and others, and they serve many uses to ease discomfort and provide soothing sensations.

A particular witch hazel extract (WH) in the embodiments, determined to provide beneficial attributes, is marketed over the counter as a product named whISOBAX™ available from Staph-Off Biotech, Inc. at staphoff.com, as example. The whISOBAX™ product comprises about 12.66 mg of gallic acid equivalent/ml. The planktonic minimum bactericidal concentration/minimum inhibitory concentration (MBC/MIC) for whISOBAX™ is about 0.31/0.15 mg/ml gallic acid equivalence method (GAE) and the minimum inhibitory concentration (MIC) for biofilm trapped bacteria is about 0.47 mg/ml GAE [~3X higher than the planktonic level].

Determined to be a particular anti-microbial component of the witch hazel extracts is the hamamelitannin molecule. The hamamelitannin molecule is substantially as follows:

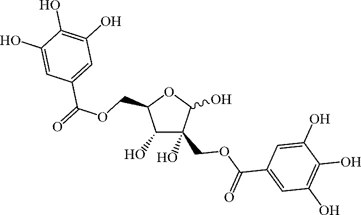 [SMILES: O=C(OC[C@H]1OC(O)[C@@](O)(COC(=O)c2cc(O)c(O)c(O)c2)[C@@H]1O)c1cc(O)c(O)c(O)c1]

Various analogues of hamamelitannin are possible and all are contemplated as suitable for the combinations in embodiments. Hamamelitannin acts as a quorum signaling inhibitor (QSI) that suppresses microbial biofilm formation and toxin production of bacteria.

Other anti-microbial active components of witch hazel include gallic acid and other phenolic compounds. Gallic acid, for example, may prevent bacterial growth by binding/disrupting cell membranes. The various components of witch hazel extract are effective in multiple gram negative and positive bacteria that commonly colonize indwelling urinary catheters and the like. The primary function of witch hazel extract where deposited and in antimicrobial actions is to block microbial attachment to devices or biological surfaces, block biofilm formation, erode existing biofilm and block microbial toxins from being produced, and lower the MIC of other anti-microbial agents.

In certain embodiments, combinations of peracetic acid and witch hazel extract concentrate are found to be particularly effective antimicrobial compositions. The acidic aqueous solution is more efficacious than individual components in limiting microbial growth. Testing of the combination of aqueous ingredients reveals that no reactants, such as precipitates, flocculates, or color change (other than dilution), results.

In certain further embodiments, increasing the concentration and acidity of either or both peracetic acid and hydrogen peroxide, while simultaneously minimizing microbe protective biofilm formation with WH, furthers antimicrobial efficacy of the peracetic acid combinations. In the combination of aqueous peracetic acid and hydrogen peroxide with WH, the WH prevents and erodes microbe induced biofilm on cellular and prosthetic surfaces.

Exceptional bench-top testing results have been obtained in antimicrobial action from the peracetic acid and WH combination in the testing of multiple microbial species. As example, the following lab test results were obtained with three concentrations of combinations of peracetic acid and WH, with hydrogen peroxide, with exemplary results for three common microbes:

| H2O2 → 6 mL | | H2O2 → 3 mL | | H2O2 → 6 mL | |
|---|---|---|---|---|---|
| whISOBAX → 1 mL | | whISOBAX → 1 mL | | whISOBAX → 1 mL | |
| Acetic acid → 1.5 mL | | Acetic acid → 1.5 mL | | Acetic acid → 5.0 mL | |
| pH → 3.5 | | pH → 3.5 | | pH → 2.5 | |
| *No reactivity observed upon initial agent mixture or three hours later | | | | | |
| **No reactivity observed at 24 hours at 37° C. incubation | | | | | |
| Staphylococcus aureus | | | | | |
| Formula 1 | | Formula 2 | | Formula 3 | |
| 1:1 | No Growth | 1:1 | No Growth | 1:1 | No Growth |
| 1:100 | No Growth | 1:100 | No Growth | 1:100 | No Growth |
| 1:1000 | No Growth | 1:1000 | No Growth | 1:1000 | No Growth |
| Escherchia coli K-12 | | | | | |
| Formula 1 | | Formula 2 | | Formula 3 | |
| 1:1 | No Growth | 1:1 | No Growth | 1:1 | No Growth |
| 1:100 | No Growth | 1:100 | No Growth | 1:100 | No Growth |
| 1:1000 | No Growth | 1:1000 | No Growth | 1:1000 | No Growth |
| Proteus vulgaris | | | | | |
| Formula 1 | | Formula 2 | | Formula 3 | |
| 1:1 | No Growth | 1:1 | No Growth | 1:1 | No Growth |
| 1:100 | No Growth | 1:100 | No Growth | 1:100 | No Growth |
| 1:1000 | No Growth | 1:1000 | No Growth | 1:1000 | No Growth |

Furthermore, example lab test results of comparison of the peracetic acid+WH, in hydrogen peroxide, combination (identified in the table as Bug-Off™), to a Betadine solution as antimicrobial follow:

| Organism | Dilution | Bug-Off™ | Betadine |
|---|---|---|---|
| Staphylococcus aureus | 1:10 | Susceptible | Susceptible |
| | 1:20 | Susceptible | Susceptible |
| | 1:40 | Susceptible | Susceptible |
| | 1:100 | Susceptible | Susceptible |
| | 1:200 | Susceptible | Resistant |
| | 1:400 | Susceptible | Resistant |
| | 1:1000 | Susceptible | Resistant |
| | 1:1500 | Susceptible | Resistant |
| Enterococcus faecalis | 1:10 | Susceptible | Susceptible |
| | 1:20 | Susceptible | Susceptible |
| | 1:40 | Susceptible | Susceptible |
| | 1:100 | Susceptible | Intermediate |
| | 1:200 | Resistant | Resistant |
| | 1:400 | Resistant | Resistant |
| | 1:1000 | Resistant | Resistant |
| | 1:1500 | Resistant | Resistant |
| Proteus mirabilis | 1:10 | Susceptible | Resistant |
| | 1:20 | Susceptible | Resistant |
| | 1:40 | Susceptible | Resistant |
| | 1:100 | Susceptible | Resistant |
| | 1:200 | Resistant | Resistant |
| | 1:400 | Resistant | Resistant |
| | 1:1000 | Resistant | Resistant |
| | 1:1500 | Resistant | Resistant |
| Klebsiella pneumoniae | 1:10 | Susceptible | Susceptible |
| | 1:20 | Susceptible | Susceptible |
| | 1:40 | Susceptible | Resistant |
| | 1:100 | Susceptible | Resistant |
| | 1:200 | Susceptible | Resistant |
| | 1:400 | Susceptible | Resistant |
| | 1:1000 | Susceptible | Resistant |
| | 1:1500 | Susceptible | Resistant |

It is noteworthy that all microbe strains, with the exception only of highest concentrations of Enterococcus faecalis and Proteus mirabilis, are more susceptible to prevention by the peracetic acid+WH+hydrogen peroxide combination (i.e., of Bug-Off™) than the conventional Betadine solution.

In use of the peracetic acid+WH+hydrogen peroxide combination, it has been the practice to obtain acetic acid and hydrogen peroxide, react the two compounds at time of use to obtain peracetic acid and remaining hydrogen peroxide, and to combine with WH. Thus, the three ingredients - hydrogen peroxide, acetic acid and WH - provide the peracetic acid+WH+hydrogen peroxide combination that has exceptional antimicrobial results. Means of mixing and dispensing are under study, such that the hydrogen peroxide and acetic acid react at point of use to yield the peracetic acid+hydrogen peroxide for combination with the WH at that point of use.

The combined peracetic acid+WH+hydrogen peroxide may be employed on surfaces of medical devices, such as for non-exclusive example Foley or other catheters, internally within organs, such as for non-exclusive example entry to the bladder, and otherwise, such as on surfaces, skin, implements and others, in the medical and microbe prevention contexts.

### Other Additives

Silver, and other metals, and halogens may be additionally or alternately included in the various combinations. These additives may have enhanced antimicrobial effectiveness in the presence of a quorum-signaling inhibitor [e.g., witch hazel components] or the like. Of course, other components may be additionally or alternately included in various combinations, such as for desired pH, toxicity, emulsifiers, compounders, and other characteristics.

The foregoing components and combinations are employable as surface coatings, for example, on the skin, or on the surface of temporarily or permanently implanted devices, and/or as and as liquid aqueous additives to surface of temporary replaceable devices like urinary catheters, and as a cleansing or rinse agent on biological organs or biological or inanimate surfaces or the like. In particular, the combinations of peracetic acid with WH are particularly effective as antimicrobials on surfaces of urinary catheters and other devices, as well as skin and wounds. The various combinations appear to be more effective as antimicrobials than would be expected from results of each component acting alone or from other antimicrobials, such as Betadine or other.

Patients with indwelling catheters, for non-exclusive example, may receive at least one or twice daily [AM/PM] instillations of a peracetic acid and witch hazel combination, with or without additional hydrogen peroxide, as a rinse into the urinary bladder for at least two days and possibly for the duration of use of the indwelling catheter (or other device, as applicable). A combination of about 5 ml to about 6 ml of about 1% to about 3% peracetic acid plus about 1 ml to about 2 ml of witch hazel may be added to the bladder lumen. Following each instillation, bladder drainage will be capped/clamped to tolerance. Unclamping and drainage of bladder urine into collection bags will be initiated by the patient or caregiver when symptoms dictate a need for urine drainage. In other applications, the above agent delivery methodology could also be effective as a twice daily dental cleansing methodology.

A urine specimen will be collected each morning from the patient’s indwelling catheter. The indwelling and/or the continuous draining catheter may be clamped for 10-15 minutes prior to urine collection if needed. After the urine specimen is collected for urine culture testing, the indwelling catheter will be irrigated with 20+ml of sterile water or saline to remove any mucous, blood, blood clots or crystalline debris from the bladder lumen. This irrigant will be discarded. Promptly thereafter, an applicable combination additive will be instilled into the bladder lumen and the catheter will be clamped to tolerance to allow the additive to remain in the bladder lumen for multiple hours. This combination additive instilled into the bladder is to remain within the bladder until the patient has urge to urinate. The patient or caregiver will then unclamp/uncap drainage tube and allow the bladder to drain into a commode or collection bag. There is no need for additive placement into the bladder lumen except for the early morning and bed-time time periods at this time. Increase or decrease in the number and timing of daily bladder instillations may be variable according to implementation. Each of the collected urine specimens will have a label for patient ID #, date, time of specimen collection and earlier time that additive was added to the bladder lumen. Additional urine specimen(s) may or may not be collected for infection testing if symptoms prompt such concerns. The specimens will be refrigerated in a research refrigerator at the local institution and then transported to a certified lab for microbiology testing.

As further protocol detail, an initial screening visit will be held. Informed consent and demographic date will be obtained. All antibiotics will be stopped at least two days prior to urine collection for the pre-treatment urine culture and for duration of the protocol procedure.

A second screening visit will then be held at least two days after the initial screening visit. A urine culture will be performed at this second screening visit.

Thereafter, in days 1, 2, and 3, after beginning urine culture from the second screening,
a. A urine dipstick test will be performed every morning.
b. The urine collection bag will be emptied prior to each bladder instillation.
c. The applicable combination shall be injected into and through the indwelling Foley catheter into the bladder in the morning and in the evening. Following instillation, the catheter will be capped, clamped or sealed in order to keep the additive within the bladder until the bladder is uncomfortably full.
e. All patients will be encouraged to keep the catheter clamped to tolerance after instillation of above fluids.
f. 10+ cc of urine will be collected directly from the indwelling bladder catheter each morning per day immediately before installation of the morning or evening combination agent. Early in day 1 collection will be made of urine specimen collection #1, followed by installation #1, and a second urine specimen collection #2 followed by installation #2 shall be made in the early AM of day 2. Third and fourth installations of the combination shall similarly be made on days 3 and 4 (mornings) Daily side effects will be logged and discussed with the patient. Single early AM bladder instillations are anticipated to eradicate the original microbial growth in the bladder urine. If single early AM instillations of the bladder additive do not sterilize bladder urine then twice daily instillations of the additive [i.e. early sunrise AM and late sunset PM] shall be offered.

Non-exclusive embodiments may take form of liquid, gel, or device coating or the like, for application to surfaces of devices or body organs or spaces or cavities for rinse or cleansing of the body or device. Additionally, the individual agents and/or combinations can be impregnated or disposed in or on tape, cloth, device, or other surfaces. The combination can be placed as a coating on devices, such as a catheter or other medical device made of polyethylene, silicone, composites, or other materials. Variations of viscosity and flow characteristics, as well as elution and retention properties, are possible in the embodiments. In certain non-exclusive alternatives, combinations of the embodiments are introduced by instillation into bodily cavities, such as bladder lumen, or other vessels or other body parts, or as coating or deposited ‘additive’ on body part, coating or deposition, as on a medical device, such as a urinary catheter or other device, or as topical application to the skin or bodily area, cavity or organ.

Of course, a wide variety of other variations are possible.

In the foregoing, therefore, the invention has been described with reference to specific embodiments. One of ordinary skill in the art will appreciate, however, that various modifications, substitutions, deletions, and additions can be made without departing from the scope of the invention. Accordingly, the specification and figures are to be regarded in an illustrative rather than a restrictive sense, and all such modifications substitutions, deletions, and additions are intended to be included within the scope of the invention. Any benefits, advantages, or solutions to problems that may have been described above with regard to specific embodiments, as well as device(s), connection(s), step(s) and element(s) that may cause any benefit, advantage, or solution to occur or become more pronounced, are not to be construed as a critical, required, or essential feature or element.

What is claimed is:

1. A biological or inanimate surface cleansing, rinsing or coating antimicrobial composition, comprising:
peracetic acid; and
witch hazel extract.

2. The composition of claim 1, further comprising:
hydrogen peroxide.

3. A reactive composition, comprising:
acetic acid; and
hydrogen peroxide;
wherein the combination reacts to form peracetic acid, and oxygen gas allowed to escape.

4. The composition of claim 3, further comprising:
witch hazel extract.

5. A method of preventing microbial proliferation, comprising:
providing acetic acid;
providing hydrogen peroxide;
reacting the acetic acid and the hydrogen peroxide to obtain peracetic acid;
providing witch hazel extract to the peracetic acid.

6. The composition of claim 4, further comprising:
witch hazel extract.

7. A process for treating the surface of a medical device, comprising:
exposing the medical device to a combination of peracetic acid and witch hazel extract.

8. A process for treating an organ, comprising:
exposing the organ to a combination of peracetic acid and witch hazel extract.

9. A medical device manufactured by the method of claim 7.

10. An organ treated by the process of claim 8.
